(19) United States
(12) Patent Application Publication
(10) Pub. No.: US 2022/0304284 A1
(43) Pub. Date: Sep. 29, 2022

(54) CONTROL AND MONITORING SYSTEM FOR PESTICIDE AND HARMFUL BEE DRUGS IN BEE HIVES

(71) Applicant: SBS BILIMSEL BIO ÇÖZÜMLER SANAYI VE TICARET A.S., Istanbul (TR)

(72) Inventors: Asli Elif TANUGUR SAMANCI, Istanbul (TR); Taylan SAMANCI, Istanbul (TR)

(21) Appl. No.: 17/434,631

(22) PCT Filed: Dec. 16, 2020
(86) PCT No.: PCT/TR2020/051306

§ 371 Date: Aug. 27, 2021

(30) Foreign Application Priority Data
Sep. 9, 2020 (TR) 2020/14298

(51) Int. Cl. A01K 47/06; G01N 33/00

(52) U.S. Cl. CPC A01K 47/06; G01N 33/0004

(57) ABSTRACT

A hive monitoring system that determines any change in chemicals (pesticide, antibiotics, bee medicines) in the hive, measures environmental and informs the control authorities so as to provide a fully monitored and controlled production from hive to spoon. There is a chemical substance sensor in the system that measures odors at levels that cannot be detected by the human nose, and records the chemical substance data with a time stamp. The measurement data is compared by a chip with the harmful bee pesticides and limit values that should be in the hives, and information is sent to the processor unit if the limit values obtained as a result of machine learning are exceeded. The users can receive the warning/informative notifications instantly via access devices over a server that receives and stores the data sent from the processor unit and can follow the conditions of the hive when desired.

### TECHNICAL FIELD

The present invention relates to a hive monitoring system that determines any change in each chemical (pesticide, antibiotics, bee medicines) in the hive, measures the environment data and informs the control authorities so as to provide a fully monitored and controlled production from the hive to the spoon.

### STATE OF THE ART

In beekeeping worldwide, diseases such as European Foul Brood, American Foul Brood, Nosema, Varrao parasite etc. are seen widely. Beekeepers use effective drugs against these diseases routinely so as to protect their bees from these diseases. Moreover, pesticide application is made to the cultivated plants where the beehives are positioned. However, there are availability limits of these drugs in the bee products worldwide. Therefore, both the dose of drugs applied by beekeepers and the amount of contamination of products such as honey, pollen, propolis, royal jelly in beehives of pesticides applied externally to herbal production is very important. For this reason, both the physical, chemical and geographical conditions are required to be appropriate and the harmful drug residues such as pesticide and antibiotic etc. to which the hives are exposed must be below the specified limit values so as to provide the bee products to comply with the quality standards.

In the hive control devices already exist in the current systems, only temperature and humidity control can be performed, information regarding bee movements and the location of the hive can be received. Moreover, the amount of nectar that comes to the hive can be measured by locating a scale under the hive. In the current systems, there are also automatic feeding systems, Heating and cooling systems that can intervene in the hive partially are available, when the critical values defined for the sensors are exceeded in these systems. However, there is no such an application that notifies the owner of the hive via e-mail or SMS when the critical values are exceeded.

Remote monitoring feature is given to the abnormal sounds in the hive with the sounds sensors that are added individually to these sensors however the transmission of the information obtained from the sensors in the hive is realized directly through the cables connected to the computer. Thus, use of said inventions in real life is not practical. In the current hive control devices, there are no sensors and systems that perform notification, can be able to measure the amount of the drugs and external pesticides applied to the beehives. Moreover, the state of the art could not be followed and self-learning artificial intelligence models could not be applicable to the current hive control systems.

In the state of the art, U.S. Pat. No. 7,549,907B2 relates to a system that records and analyzes the honey bee sounds so as to monitor the hive health. The recorded sound data are filtered and analyzed by means of software, the determination and detection of the toxic substances carried by airway is provided. The bees make different sounds as a result of the non-lethal toxic substances to which they are exposed. The hive health can be determined as a result of the analysis of the sounds made by the bees. It is not possible to detect toxic substances exactly and whether they are within the limit value or not although they can be determined. Moreover, it is not possible to reach the instant data as well as to monitor the detected information remotely.

As a result, due to the abovementioned disadvantages and the insufficiency of the current solutions regarding the subject matter, a development is required to be made in the relevant technical field.

### AIM OF THE INVENTION

The invention covers a system to be used by packaging food companies of the bee products that serve in the beekeeping sector, beekeeper unions, honey producer unions, the ministry of agriculture and forestry, and directorates of provincial and district agriculture. Each chemical, physical and geographical change in the hive can be monitored with this system from the hive to the spoon preferred by the companies that intend to provide fully monitorable and controllable production. Moreover, a smart control system will be provided with which the producer and control authorities can control the quality of the product with a timely response. Moreover, public institutions and beekeepers and associations of honey producers may prefer this so as to control the production of the quality of bee products of the beekeepers.

The drug applications, health of the bees, presence of the colony and mobility of the hives will be managed and monitored with the invention as well as determinations are made regarding drugs placed in the hives such as pesticide and antibiotic to which the bee colonies are exposed. At the same time, aroma and health condition in the hive will be monitored with the algorithms of machine learning by measuring the gas composition within the environment by means of an electrochemical (electronic nose) device.

Another aim of the invention is to collect all data in an environment with the wireless communication and internet of things (IoT) logic by placing sensors in the hive and to provide this data to be learnt by the machine. Therefore, the artificial intelligence will not only distinguish the pesticides used in the hive but also will keep the data regarding when the drugs or external pesticide contamination occurred, how much they are used etc. in its memory and will provide the meaningful data to the users. The users can take correct and suitable precautions according to the obtained data.

In addition to the abovementioned features, the invention comprises the current methods in its structure and in the device placed in hive, it provides to remotely control the hive by means of the sensors that can be able to measure/count the sound level of the bees, in hive humidity level, in hive temperature, the mobility of the hives, the location, inlet and outlet of the bees, the number of opening and closing of the hive.

It is provided to monitor the stress conditions such as hunger, swarming, absence of queen bee by measuring the humidity and temperature in hive by means of measuring the sound level of the bees with the sound sensor. The inlet and outlet of the bees are counted with the camera placed in front of the hive inlet door and thus information regarding the nectar arrival condition is provided by determining the number of flying bees. The communication of the low power RFID module and the external unit with the in hive device is provided. In hive GPS module provides monitoring the hives.

The inventive system provides to send message and/or e-mail to the mobile phones, tablets in case of any limit exceeding with the help of the lower and upper limits determined for the sensors in the hive.

The structural and characteristic features of the invention and all its advantages will be understood clearly by the following drawings and the detailed description made with reference to these drawings.

### DESCRIPTION OF THE FIGURES

FIG. 1, is a view of the inventive hive monitoring system.

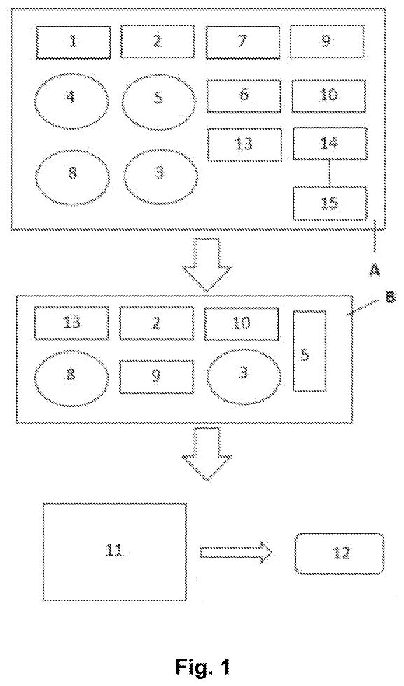

The figures are not required to be scaled and the details which are not necessary for understanding the present invention may be neglected.

### DESCRIPTION OF THE PART REFERENCES

A, In hive unit
B. Out hive unit
1. Sound sensor
2. Humidity sensor
3. Memory unit
4. Cover sensor
5. GPS module
6. Count sensor
7. Motion sensor
8. Processor unit
9. Communication module
10. Battery
11. Server
12. Access device
13. Temperature sensor
14. Chemical substance sensor
15. Chip

### DETAILED DESCRIPTION OF THE INVENTION

In this detailed description, the preferred embodiments of the invention is described only for clarifying the subject matter in a manner such that no limiting effect is created.

There are 2 units in the inventive hive monitoring system as internal and external. In the preferred embodiment, in the out hive unit (B); there are the following; processor unit (8), memory unit (3), GPS module (5), humidity sensor (2), temperature sensor (13), (RFID) communication module (9) and battery (10). In the preferred embodiment, in the in hive unit (A); there are the following; processor unit (8), memory unit (3), humidity sensor (2), sound sensor (1), hive cover sensor (4), GPS module (5), bee count sensor (6), movement sensor (7), temperature sensor (13), chemical substance sensor (14), chip (15), communication module (9) and battery (10).

Learning process with the learning module is applied for the operation of the invention. The operations performed at this phase are as follows;
The learning module is trained for a certain period (3-6 months) in sample apiary in order to control with wireless communication.
In this apiary, hives that are good, very good, bad, very bad, healthy and unhealthy etc. are maintained and hives are intentionally dragged into environments such as excess, low, medium antibiotics, pesticides and harmful bee drugs.
The whole process is sent to the data center via the Internet of Things. The learning module learns the condition of the antibiotics, pesticides and harmful bee drugs used in the hives by making a comparison between the received data and the hives with already known conditions.
The data regarding the hive are also supported by the analysis data obtained manually.
The quality of honey obtained in the hives is determined and confirmed by melissopalynological, physico-chemical, pesticide and antibiotic analyses.

After the learning process is completed, the learning module is integrated into the chip (15) connected to the chemical substance sensor (14) with integration and placed in the hives. Therefore, the artificial intelligence in the chip (15) measures the harmful bee drugs such as antibiotic and pesticide required to be available in the hives and applies the required processes in case the limit values are exceeded. The chemical substance sensor (pesticide—antibiotic, odor electronic nose) (14) located in the hive performs precise measurement with the chemical sensor sequence in its structure on the odors at such levels that cannot be detected with human nose. Data regarding residue, aroma etc. in the hives are collected with this sensor. The odors within the ambient received by means of the chemical substance sensor (14) are processed in the chip (15) with the machine learning and classification studies are carried out. Condition of exceeding the limit values is monitored by making the analysis of the pesticide, antibiotic and other harmful drugs applied in the hive and it is monitored whether veterinary medicine application is carried out or not in the hive.

Also measuring sound sensor (1), humidity sensor (2), cover sensor (4), count sensor (6), motion sensor (7), temperature sensor (13) are added so as to monitor the hive remotely. The sound levels generated by the bees in the hive are measured so as to monitor the hunger and swarming conditions by means of the sound sensor (1), The humidity sensor (2) in the in hive and out hive units (A, B) measures the humidity of the internal and external air. The cover sensor (4) located in the in hive unit (A) provides to determine how many times the hive is opened for maintenance by counting the number of times the upper cover of the hive is opened and closed or by whom the cover is opened other than the beekeeper. The count sensor (6) in the in hive unit (A) provides to determine the number of flying bees by means of counting the inlet and outlet of the bees to/from the hive and to obtain information about the nectar arrival condition. The motion sensor (7) is the sensor that detects the movements of the hive, determines the conditions as the overturn of the hive or the occurrence of an impact. The temperature sensor (13) in the in hive and out hive units (A, B) measures the temperature of the internal and external air. All these sensor data collected are sent to the memory units (3) and processor units (8).

The data received from the chemical substance sensor (14) are sent to the memory unit (3) attached to the beehive with time stamp and hive number. Open source coded systems are used so as to save the data. The Influx open source based database management system that exhibits good performance in preferably IoT and time stamped records is used among these. The processor unit (8) located in the in hive unit (A) sends the information received from all sensors in the hive to the out hive unit (B) or to the access devices (12) of the users directly by means of the communication (GSM) module (9) as a notification. A warning/informative message and/or e-mail is sent to the access device (12) that can be a mobile device, computer or an alternative access device preferred by the user in case of any limit exceeding by determining lower and upper limits for all sensors. It is provided that the out hive unit (B) keeps the information received from the in hive unit (A) in the memory unit (3) or sends the same to the server (11) directly. The data received from the sensors present in the external unit (B) are sent to the server (11). The data is preferably transmitted to the software in the server (11) over the cloud. The server (11) provides the instant transmission of the warning/informative notifications (SMS and/or e-mail) to the access device (12) (mobile device, computer etc.) that the user prefers. Thanks to aforementioned software, the beekeeper and the institution managing the system can be able to control the condition of the hives by entering this software (portal) with the password and user name belong to them via the access device (12). There are applications of the system that can operate on the Android and ICES operating systems in the mobile phones and tablets.

There are batteries (10) in the hive monitoring system that provide the required energy so as to operate the elements in the in hive and out hive units (A, B), Preferably, the batteries (10) used are the energy sources that are operating with long-lasting battery or solar energy. The location of the hive is determined and monitoring of the hive is provided by an access device (12) by means of the GPS module (5) that is located in the out hive unit (B) and can be able to send data with the SIM card inserted therein.

1. A hive monitoring system for monitorable and controllable honey production, the system comprising:
a chemical substance sensor located in an in hive unit that has a chemical sensor sequence in its structure and makes measurement on odors at levels that cannot be detected by the human nose, records the chemical substance data with a time stamp,
a chip having artificial intelligence that compares the measurement data obtained by the chemical substance sensor and limit values of the required harmful bee drugs that must be present in the hives, sends information to a processor unit in case the limit values obtained as a result of the training with machine learning is exceeded;
a processor unit that sends the raw and/or processed data in the in hive and out hive units by means of a communication module;
a server that receives and stores the data sent from the processor unit and transmits warning/informative notifications to a user's access device instantly;
a battery that provides required energy so as to operate the elements in the in hive and out hive units.

2. The hive monitoring system according to claim 1, comprising a sound sensor that is located in the in hive unit, the sound sensor measuring sound levels generated by the bees in the hive so as to monitor hunger and swarming conditions.

3. The hive monitoring system according to claim 1, comprising humidity sensors in the in hive and out hive units that measures humidity of the internal and external air.

4. The hive monitoring system according to claim 1, comprising a cover sensor that is located in the in hive unit, the cover sensor configured to count the number of opening and closing of an upper cover of the hive.

5. The hive monitoring system according to claim 1, comprising a motion sensor that is located in the in hive unit, the motion sensor configured to detect movements of the hive.

6. The hive monitoring system according to claim 1, comprising temperature sensors in the in hive and out hive units that measure the temperature of the internal and external air.

7. The hive monitoring system according to claim 1, comprising a GPS module that is located in the out hive unit and which is configured to send data with a SIM card inserted therein, and which provides for determining the location of the hive and monitoring the same via an access device.

8. The hive monitoring system according to claim 1, comprising a count sensor that is located in the in hive unit, the count sensor configured to determine the number of flying bees by counting the inlet and outlet of the bees to/from the hive and to obtain information about a nectar arrival condition.

9. The hive monitoring system according to claim 1, comprising a memory unit in which the raw and/or processed data obtained in the in hive and out hive units are stored.
